(19) United States
(12) Patent Application Publication
(10) Pub. No.: US 2017/0020946 A1
(43) Pub. Date: Jan. 26, 2017

(54) ANALGESIC COMPOSITIONS AND METHODS OF USE

(71) Applicant: William T. Cozart, Lenoir, NC (US)

(72) Inventor: William T. Cozart, Lenoir, NC (US)

(21) Appl. No.: 15/285,168

(22) Filed: Oct. 4, 2016

Related U.S. Application Data
(63) Continuation of application No. 14/689,077, filed on Apr. 17, 2015, now abandoned.
(60) Provisional application No. 61/981,534, filed on Apr. 18, 2014.

(51) Int. Cl. A61K 36/61; A61K 31/245; A61K 47/02; A61K 47/10; A61K 9/06; A61K 36/9068; A61K 36/534; A61K 36/28; A61K 36/63; A61K 36/87; A61K 45/06; A61K 9/00

(52) U.S. Cl. CPC A61K 36/61; A61K 45/06; A61K 31/245; A61K 47/02; A61K 47/10; A61K 9/006; A61K 36/9068; A61K 36/534; A61K 36/28; A61K 36/63; A61K 36/87; A61K 9/06

(57) ABSTRACT

The invention relates to the fields of pharmacology and medicine, and provides anesthetic compositions and methods for the treatment of pain using compositions comprising Tea tree oil and one or more additional agent. The invention relates to analgesic compositions for the treatment of pain associated with a variety of disorders or conditions, particularly oral or dental disorders or conditions such as alveolar osteitis or dental caries.

### CROSS REFERENCE TO RELATED APPLICATIONS

This application claims priority to U.S. Provisional Patent Application No. 61/981,534, filed Apr. 18, 2014, which is hereby incorporated by reference in their entireties.

### FIELD OF INVENTION

The invention relates to the fields of pharmacology and medicine, and provides anesthetic compositions and methods for the treatment of pain using compositions comprising Tea tree oil and one or more additional agent. In particular, the invention relates to analgesic compositions for the treatment of pain associated with a variety of disorders or conditions, particularly oral or dental disorders or conditions such as alveolar osteitis or dental caries.

### BACKGROUND

Pain can be defined as an unpleasant sensation ranging from mild discomfort to agonizing distress, associated with real or potential tissue damage, or a disorder of the nervous system. Pain is a response to impulses from the peripheral nerves in damaged tissue, which pass to nerves in the spinal cord. All animals experience some degree of pain during life, whether through injury or disease. As such, one of the major areas of drug research is the development of analgesics to be used in pain management.

Alveolar osteitis (also known as “alveolitis sicca dolorosa” or “dry socket”) is the most common complication following a tooth extraction, particularly from extraction of third molars. Other synonymous terms are “localized osteitis”, “postoperative alveolitis”, “alveolalgia”, “septic socket”, “necrotic socket”, “localized osteomyelitis”, and “fibrinolytic alveolitis”. The incidence of alveolar osteitis has been reported as 3-4% following routine dental extractions and ranges from 1% to 45% after the removal of mandibular third molars. The incidence of dry socket is higher in the mandible, occurring up to 10 times more often for mandibular third molars compared to maxillary molars.

Alveolar osteitis is physically characterized by an empty tooth socket with exposed bone surfaces surrounded by inflamed tissue. The denuded alveolar bone results in extreme pain, irradiating from the empty socket, normally to the ipsilateral ear, temporal region or the eye. The clinical presentation of alveolar osteitis includes post-operative pain in and around the extraction site, which increases in severity at any time between 1 and 3 days after the extraction. Post-operative pain is accompanied by a partially or totally disintegrated blood clot within the alveolar socket, with or without halitosis. Pathogenesis of alveolar osteitis appears to result from the conversion of plasminogen to plasmin resulting in fibrinolysis of the blood clot within the extraction socket.

Factors attributed to the disruption in the healing of the extraction wound include trans-operative complications, presence of local infection, bacterial contamination of the socket, experience of the surgeon, contraceptive use, smoking, alcohol intake, and use of local anesthetics with vasoconstrictors. Individuals with diabetes mellitus, hormonal imbalances, antibiotics-induced immunosuppression, chemotherapy-induced immunosuppression, AIDS-related immunosuppression, and radiation therapy-induced immunosuppression are especially susceptible to problems in the healing of the extraction wounds. Other factors such as smoking, excessive trauma to the tissue site, degree of impaction of the third molar, inadequate irrigation during and after extraction, oral conceptive use, timing in the menstrual cycle, use of an anesthetic with a vasoconstrictor, use of corticosteroids preoperatively, extraction-associated surgical trauma, and experience of the oral surgeon all have been identified as probable causes. Additional risk factors include presence of pericorontitis, high pre- and post-operative bacterial counts.

Regardless of cause, dry socket remains a prevalent and painful condition. Over the years many different materials and methods have been used for treating alveolar osteitis. None have been particularly effective.

Dental caries is also a common disease process that afflicts a large proportion of the world population. Extensive research indicates that dental caries is the result of a bacterial infection (Loesche (1986) Microbiol. Rev. 50:353-380), but is also influenced by host and dietary factors (Hicks et al. (2003) J. Clin. Pediatr. Dent. 28:47-52). Current research seeks to identify risk factors for caries and to identify natural oral defenses that may protect against or prevent caries development. Salivary defense systems play a significant role in maintaining the health of the oral cavity and preventing caries. These defenses include factors which inhibit or reverse demineralization of exposed tooth surfaces, such as simple mechanical rinsing, buffering action, and calcium phosphate binding proteins, as well as antimicrobial activities including microorganism aggregation and clearance from the oral cavity, immune surveillance, and the secretion of antimicrobial peptides (Van Nieuw Amerongen et al. (2004) Caries Res. 38:247-253).

Severe dental caries is associated with severe pain until the subject is able to receive treatment. Lidocaine is a local anaesthetic of the amide type that was first introduced in the 1940s and is widely used in injection and for local application to mucous membranes. It has rapid onset of action and anesthesia is obtained within a few minutes depending on the site of administration. It has an intermediate duration of action, with an elimination half-life of approximately 90-120 minutes in most subjects. Benzocaine is an ethyl ester of p-Aminobenzoic acid and is used to relieve pain associated with ulcers, wounds, mucous membranes, and various types of oral and dental pain. Normally benzocaine acts only as long as it is in contact with the skin or mucosal surface, with a peak effect occurring within 1 minute after application and lasting for 30 to 60 minutes.

Accordingly, there is a need for improved long-lasting analgesic compositions for the treatment of pain, particularly pain associated with oral or dental disorders or conditions such as alveolar osteitis or dental caries.

### SUMMARY

An anesthetic composition is provided comprising a combination of Tea tree oil and one or more additional agent. In a particular embodiment, an anesthetic composition is provided comprising a combination of Tea tree oil and one or more additional agent, particularly comprising 0.1 ml to 1.0 ml of Tea tree oil. In another particular embodiment, the ratio of Tea tree oil to the one or more additional agent is about a 6:1 part by weight relationship. Additional agents may be selected from the group consisting of: Ginger root oil, Peppermint leaf oil, Chamomile flower oil, Olive leaf extract oil, Grape seed oil, Clove oil, a flavoring syrup (e.g., Cherry syrup), and an additional anesthetic agent (e.g., a topical anesthetic such as benzocaine, lidocaine, and/or tetracaine). In one embodiment, the anesthetic composition comprises a combination of Tea tree oil, Ginger root oil, Peppermint leaf oil, Chamomile flower oil, Olive leaf extract oil, Grape seed oil, Clove oil, a flavoring syrup, and an additional anesthetic agent in a ratio of about a 6:1:1:1:1:1:1:1:1 part by weight relationship. In a particular embodiment, the additional anesthetic agent is a topical anesthetic, particularly benzocaine. In a further embodiment, the anesthetic composition may further comprise pharmaceutically acceptable solvents, diluents, and/or carriers. In a particular embodiment, the anesthetic composition may further comprise zinc oxide and/or water soluble poly(ethyleneglycol) (PEG) such as PEG 3350 and/or PEG 400.

In one embodiment, the composition is in paste form. In other embodiments, the composition may be formulated as a gel or liquid. As compared to conventional anesthetics, the present anesthetic composition has improved efficacy, potency, and duration when applied to treat pain.

In another embodiment of the presently disclosed subject matter, the anesthetic composition is administered to a subject in need thereof in a therapeutically effective amount to treat pain associated with abrasions, rashes, cuts, burns, cold sores in and out of the mouth, canker sores, mouth sores such as those caused by surgery or cancer treatment with chemotherapy or radiation therapy, surgical wounds, diabetic sores or ulcers, or pressure sores. In another embodiment, the anesthetic composition is administered to a subject in need thereof in a therapeutically effective amount to treat pain associated with an oral or dental disorder or condition, particularly alveolar osteitis or dental caries.

In another embodiment, kits are provided comprising an analgesic composition as presently disclosed along with instructions for use in treating pain associated with abrasions, rashes, cuts, burns, cold sores in and out of the mouth, canker sores, mouth sores such as those caused by surgery or cancer treatment with chemotherapy or radiation therapy, surgical wounds, diabetic sores or ulcers, or pressure sores. In another embodiment, kits are provided comprising an analgesic composition as presently disclosed along with instructions for use in treating pain associated with an oral or dental disorder or condition in a subject in need thereof, particularly alveolar osteitis or dental caries.

Certain aspects of the presently disclosed subject matter having been stated hereinabove, which are addressed in whole or in part by the presently disclosed subject matter, other aspects will become evident as the description proceeds when taken in connection with the accompanying Examples as best described herein below.

### BRIEF DESCRIPTION OF THE DRAWINGS

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 is a photograph of teeth with severe dental caries that were removed from patients who had received treatment as described in Example 2.

### DETAILED DESCRIPTION

The presently disclosed subject matter now will be described more fully hereinafter. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

The presently disclosed subject matter is directed to anesthetic compositions and methods using compositions comprising Tea tree oil and one or more additional agent.

### I. Compositions

In one embodiment of the presently disclosed subject matter, an anesthetic composition is provided comprising a combination of Tea tree oil and one or more additional agent. In a particular embodiment, an anesthetic composition is provided comprising a combination of Tea tree oil and one or more additional agent, particularly comprising 0.1 ml to 1.0 ml of Tea tree oil. In another particular embodiment, the ratio of Tea tree oil to the one or more additional agent is about a 6:1 part by weight relationship. Additional agents may be selected from the group consisting of: Ginger root oil, Peppermint leaf oil, Chamomile flower oil, Olive leaf extract oil, Grape seed oil, Clove oil, a flavoring syrup (e.g., Cherry syrup), and an additional anesthetic agent (e.g., a topical anesthetic such as benzocaine, lidocaine, and/or tetracaine).

Tea tree oil, or Melaleuca oil, is an essential oil with a camphoraceous odor and a color ranging from pale yellow to colorless or clear. Tea tree oil is extracted from the leaves of Melaleuca alternifolla of the Myrtaceae family and is also known as ti-tree, ti-trol and melasol. Tea tree oil may be extracted from the leaves and twigs of Melaleuca alternifolla by steam distillation with a yield of about 1.8%. The chemical components of Tea tree oil include a-pinene, b-pinene, sabinene, myrcene, a-phellandrene, a-terpinene, limonene, 1,8-cineole, y-terpinene, p-cymene, terpinolene, linalool, terpinen-4-ol, and a-terpineol.

Ginger root oil, or Ginger essential oil, is extracted from the dried root (rhizomes) of Zingiberaceae officinale of the Zingiberaceae family (also known as Common or Jamaica Ginger). The oil may be extracted by means of steam distillation from the unpeeled or dried, ground-up rhizomes of the plant, and can yield about 2-4% oil. The essential oil has various chemical constituents including the following: a-pinene, camphene, b-pinene, 1,8-cineole, linalool, borneol, y-terpineol, nerol, neral, geraniol, geranial, geranyl acetate, b-bisabolene, and zingiberene.

Peppermint leaf oil, or Peppermint essential oil, is extracted from Mentha piperita of the Labiatae family and is also known as brandy mint and balm mint. Peppermint leaf oil may be extracted from the whole fresh or partly dried plant just before flowering by steam distillation with a yield of about 0.1-1.0%. The chemical components of Peppermint leaf oil include menthol, menthone, 1,8-cineole, methyl acetate, methofuran, isomenthone, limonene, b-pinene, a-pinene, germacrene-d, trans-sabinene hydrate, and pulegone.

Chamomile flower oil, also known as Roman Chamomile essential oil, is produced from Anthemis nobilis (Chamaemelum nobile) of the family Asteraceae, formerly placed in the Compositae family. It is also known as English chamomile, sweet chamomile, and garden chamomile. Roman chamomile oil may be extracted from the flowers by steam distillation to yield about 1.7% from fresh flowers. The chemical components of Roman chamomile oil include a-pinene, camphene, b-pinene, sabinene, myrcene, 1,8-cineole, y-terpinene, caryophyllene, and propyl angelate and butyl angelate.

Olive leaf extract oil, is produced from Olea europa of the family Oleaceae. The leaves contain secoiridoids, including oleuropein as well as ligustroside and oleacein. The chemical components of olive leaf extract oil include triterpenoids (oleanolic acid and uvaol), sterols, flavenoids (chrysoeriol, apigenin, and luteolin glycosides) and various other phenolic acids.

Grape seed oil is produced from Vitis vinifera. The chemical components of Grape seed oil include fatty acids such as palmitic, palmitoleic, stearic, oleic, linoleic, alpha linoleic, icosanoic, icosenoic, and docosanoic fatty acids.

Clove oil, also known as Clove essential oil, is extracted from Eugenia caryophyllata (also known as Syzygium aromaticum, Eugenia aromatica, E. carophyllus) of the Myrtaceae family. Clove oil may be extracted from the leaves, stem and buds. The chemical components of clove oil include eugenol, eugenol acetate, iso-eugenol, and caryophyllene.

A syrup is a concentrated or nearly saturated solution of sucrose in water. Syrups containing pleasantly flavored substances are known as flavoring syrups (e.g. Cherry Syrup, Acacia Syrup, etc.). For example, Cherry syrup may be produced by dissolving sucrose in cherry juice then adding alcohol and purified water.

Additional anesthetic agents may include topical anesthetics such as benzocaine, lidocaine, and/or tetracaine. Lidocaine is a local anaesthetic of the amide type that was first introduced in the 1940s and is widely used in injection and for local application to mucous membranes. It has rapid onset of action and anesthesia is obtained within a few minutes depending on the site of administration. It has an intermediate duration of action, with an elimination half-life of approximately 90-120 minutes in most subjects. Benzocaine is an ethyl ester of p-Aminobenzoic acid and is used to relieve pain associated with ulcers, wounds, mucous membranes, and various types of oral and dental pain. Normally benzocaine acts only as long as it is in contact with the skin or mucosal surface, with a peak effect occurring within 1 minute after application and lasting for 30 to 60 minutes. Tetracaine is a potent local anesthetic that is synthesized from 4-butylaminobenzoic acid. The ethyl ester is formed through an acid-catalyzed esterification reaction. Base-catalyzed transesterification is achieved by boiling the ethyl ester of 4-butylaminobenzoic acid with excess 2-dimethylaminoethanol in the presence of a small amount of sodium ethoxide.

In one embodiment, the anesthetic composition comprises a combination of Tea tree oil, Ginger root oil, Peppermint leaf oil, Chamomile flower oil, Olive leaf extract oil, Grape seed oil, Clove oil, a flavoring syrup (e.g. Cherry syrup), and an additional anesthetic agent in a ratio of about a 6:1:1:1:1:1:1:1:1 part by weight relationship. In a particular embodiment, the additional anesthetic agent is a topical anesthetic, particularly benzocaine.

In a further embodiment, the anesthetic composition may further comprise physiologically or pharmaceutically acceptable carriers. As used herein, “physiologically compatible carrier” refers to a physiologically acceptable diluent including, but not limited to water, phosphate buffered saline, or saline, and, in some embodiments, can include an adjuvant. Specifically, in a preferred embodiment, the carrier is substantially inactive, with the exception of its intrinsic surfactant properties which are used in the production of a suspension of the active ingredients. The compositions may include other physiologically active constituents that do not interfere with the efficacy of the active agents in the composition. The carriers utilized in the therapeutic compositions of the presently disclosed subject matter may be paste, liquid, or gel-based materials for use in paste, liquid, or gel formulations. The specific formulations depend, in part, upon the routes or modes of administration. Suitable paste, liquid, or gel-based carriers are well-known in the art (e.g., water, physiological salt solutions, urea, and the like). Preferably, water-based carriers have approximately neutral pH.

Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or nonionic surfactants such as Tween, Pluronics, or PEG. Adjuvants suitable for use with the presently disclosed compositions include adjuvants known in the art including, but not limited to, incomplete Freund's adjuvant, aluminum phosphate, aluminum hydroxide, and alum. Other suitable carriers include aqueous and oleaginous carriers such as, for example, white petrolatum, myristate, lanolin, mineral oil, fragrant or essential oil, nasturtium extract oil, sorbitan mono-oleate, detergents (e.g., polyoxyl stearate or sodium lauryl sulfate) and mixed with water to form a lotion, gel, cream, or paste, semi-solid composition. Additional suitable carriers comprise water-in-oil or oil-in-water emulsions and mixtures of emulsifiers and emollients with solvents such as sucrose stearate, sucrose cocoate, sucrose distearate, mineral oil, and water. For example, emulsions containing water, glycerol stearate, glycerin, mineral oil, synthetic spermaceti, butylparaben, propylparaben and methylparaben are commercially available. Preservatives may also be included in the carrier including methylparaben and propylparaben. The composition may also include a plasticizer such as glycerol. The composition of the carrier can be varied so long as it does not interfere significantly with the pharmacological activity of the active ingredients of the therapeutic composition.

The preparation of pharmaceutical formulations is well known in the art and has been described in many articles and textbooks, see e.g., Gennaro A. R. ed. (1990) Remington's Pharmaceutical Sciences, Mack Publishing Company, Easton, Pa., and especially pages 1521-1712 therein.

Compositions to be used for in vivo administration must be sterile, which can be achieved by filtration through sterile filtration membranes, prior to or following lyophilization and reconstitution. Therapeutic compositions may be placed into a container having a sterile access port, for example, an intravenous solution bag, vial, or carpule having a stopper pierceable by an injection needle.

In a particular embodiment, the anesthetic composition may further comprise zinc oxide and/or water soluble poly(ethyleneglycol) (PEG) such as PEG 3350 and/or PEG 400. In one embodiment, the composition is in paste form. In other embodiments, the composition may be formulated as a gel or liquid. As compared to conventional anesthetics, the present anesthetic composition has improved efficacy, potency, and duration when applied to treat an oral or dental disorder or condition, particularly alveolar osteitis or dental caries.

In another particular embodiment, the anesthetic composition comprises a combination of Tea tree oil and one or more additional agent, wherein the ratio of Tea tree oil to the one or more additional agent is about a 6:1 part by weight relationship. In a further particular embodiment, the additional agent is an additional anesthetic agent, particularly a topical anesthetic agent selected from the group consisting of benzocaine, lidocaine, and tetracaine. In other particular embodiments, the additional agent is one or more agent selected from the group consisting of Ginger root oil, Peppermint leaf oil, Chamomile flower oil, Olive leaf extract oil, Grape seed oil, Clove oil, and a flavoring syrup (e.g. Cherry syrup). In yet another particular embodiment, the anesthetic composition further comprises zinc oxide and/or water soluble poly(ethyleneglycol) (PEG) such as PEG 3350 and/or PEG 400.

In another embodiment, the anesthetic composition used in any of the preceding or alternative embodiments of the present invention may include agents in an analgesically effective amount between about 0.01 mg to about 500 mg per unit dose, and in particular between about 1 mg to about 100 mg per unit dose.

In another embodiment, the anesthetic composition used in any of the preceding or alternative embodiments of the present invention may be in the form of an immediate-release composition, a controlled-release composition, sustained-release orally-administrable compositions, topically-administrable compositions, liquid solutions, liquid sprays, lozenges, throat sprays, ointments, solutions, foams, cough drops, dissolvable strips, a jelly, a mouthwash, a gargle, a lollipop, a gum, aqueous or oily suspensions, dispersible powders or granules, a syrup, an elixir, emulsions, a cream, a paste, a gel, a lotion, impregnated dressings, occularly-administrable compositions, inhalable particles, inhalable solutions, droplets, or aerosols.

Accordingly, compositions of the present invention that are intended for oral use may be in the form of a pill, tablet, gelcap, or hard or soft capsule (each of which may be in an immediate, sustained or time-release formulation); lozenge; throat spray; solution; emulsion; cream; paste; gel; cough drop; dissolvable strip; lollipop; gum; aqueous or oily suspension, dispersible powder/granules; syrup; elixir; and may be prepared according to any method known in the art for the manufacture of pharmaceutical compositions. Such compositions may further contain one or more agents selected from the group consisting of sweetening agents, flavoring agents, coloring agents and preserving agents in order to provide pharmaceutically elegant and palatable preparations. By dissolvable strip is meant a sheet of material that can be placed in the mouth to dissolve and release the active ingredient or prodrug. Such dissolvable strips are also known as flavor strips or oral care strips. Dissolvable strips are often carbohydrate-based. Tablets typically contain the active ingredient or prodrug in admixture with non-toxic pharmaceutically acceptable excipients which are suitable for the manufacture of such tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example, magnesium stearate, stearic acid or talc. Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

With respect to capsules, in hard gelatin capsule formulations the active ingredient(s) may be mixed with an inert solid diluent, for example, calcium carbonate, calcium phosphate or kaolin; and in soft gelatin capsule formulations the active ingredient(s) may be mixed with water or an oil medium, for example peanut oil, liquid paraffin, or olive oil.

Aqueous suspensions typically contain the active material in admixture with excipients suitable for the manufacture of such aqueous suspensions. Such excipients may be suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents, such as a naturally-occurring phosphatide (e.g., lecithin), condensation products of an alkylene oxide with fatty acids (e.g., polyoxyethylene stearate), condensation products of ethylene oxide with long chain aliphatic alcohols (e.g., heptadecaethylene-oxycetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives, for example ethyl, n-propyl, or p-hydroxybenzoate; one or more coloring agents; one or more flavoring agents; and/or one or more sweetening agents, such as sucrose, saccharin or aspartame.

Oily suspensions may be formulated by suspending the active ingredient or prodrug in a vegetable oil, such as arachis oil, olive oil, sesame oil or coconut oil; or in mineral oil, such as liquid paraffin. Such oily suspensions may also contain a thickening agent, such as beeswax, hard paraffin or cetyl alcohol. Sweetening agents, such as those set forth above, and flavoring agents, may also be added to provide a palatable oral preparation. Such compositions may be preserved by the addition of an anti-oxidant, such as ascorbic acid.

Dispersible powders and granules suitable for preparation of an aqueous suspension by the addition of water typically provide the active ingredient or prodrug in admixture with a dispersing or wetting agent, a suspending agent and one or more preservatives. Suitable dispersing or wetting agents and suspending agents are exemplified by those aforementioned. Additional excipients, for example sweetening, flavoring and coloring agents, may also be present.

Anesthetic compositions of the presently disclosed subject matter may also be in the form of oil-in-water emulsions. In such preparations, the oily phase may be a vegetable oil (e.g., olive oil or arachis oil), a mineral oil (e.g., liquid paraffin), or mixtures of such vegetable and mineral oils. Suitable emulsifying agents may be naturally-occurring phosphatides, such as soy bean, lecithin, esters or partial esters derived from fatty acids and hexitol anhydrides (e.g., sorbitan monooleate), and condensation products of such partial esters with ethylene oxide (e.g., polyoxyethylene sorbitan monooleate). Such emulsions may also contain sweetening and flavoring agents.

Syrups and elixirs may be formulated with sweetening agents, such as glycerol, propylene glycol, sorbitol or sucrose. Such formulations may also contain a demulcent, a preservative, and flavoring and/or coloring agents.

Analgesic compositions of the presently disclosed subject matter may also be in the form of a sterile injectable aqueous or oleaginous suspension, which may be formulated according to methods known in the art using, for example, suitable aforementioned dispersing or wetting agents, and suspending agents. Such a sterile injectable preparation may also be a sterile injectable solution or suspension in a non-toxic parenterally-acceptable diluent or solvent, such as a solution in 1,3-butane diol. Acceptable vehicles and solvents that may be employed include, for example, water, Ringer's solution and isotonic sodium chloride solution. Additionally, sterile, fixed oils may be employed as a solvent or suspending medium, such as a bland fixed oil, including synthetic mono- or diglycerides. Fatty acids such as oleic acid, may also be used in the preparation of injectables.

As aforementioned, the analgesic compositions may be administered in a controlled or sustained release system. Such systems include, for example, the use of a pump (see, for example, Langer and Sefton, (1987) CRC Crit. Ref Biomed. 14:201; Buchwald et al. (1980), Surgery 88:507; Saudek et al. (1989), N. Engl. J. Med. 321:574), and more typically (with respect to oral formulations such as pills, tablets, etc.), the use of polymeric materials (see, for example, Medical Applications of Controlled Release, Langer and Wise (eds.) (1974), CRC Pres., Boca Raton, Fla.; Controlled Drug Bioavailability, Drug Product Design and Performance, Smolen and Ball (eds.) (1984), Wiley, New York; Ranger and Peppas, J. (1983) Macromol. Sci. Rev. Macromol. Chem. 23:61; Levy et al. (1985) Science 228:190; During et al. (1989) Ann. Neurol. 25:351; Howard et al. (1989) J. Neurosurg. 71:105). Other means of effecting controlled release involve, for example, placing the therapeutic composition in proximity of the therapeutic target, thus requiring only a fraction of the systemic dose (see, for example, Goodson, Medical Applications of Controlled Release, (1984) vol. 2, pp. 115-138). Other controlled release systems which may be employed include those reviewed by Langer (Science (1990) 249:1527-1533).

Analgesic compositions of the presently disclosed subject matter, may also be administered in the form of rectal suppositories. Such compositions may be prepared by mixing the active compound with a suitable non-irritating excipient which is solid at ordinary temperatures but liquid at rectal temperature, and will therefore melt in the rectum to release the active compound. Suitable rectal suppository materials include cocoa butter and polyethylene glycols.

For topical administration of the analgesic compositions, a liquid solution; liquid spray; emulsion; cream; paste; gel; lotion; foam; impregnated dressing; ointment; jelly; or mouth wash/gargle may be employed.

Analgesic compositions of the present invention may also be administered occularly, such as in the form of eye-drops, ointments, sprays or conjunctival timed-release inserts. Administration of the analgesic compositions to the sinuses, throat, or lungs may be in the form of inhalable particles, inhalable solution, droplets, inhalation sprays, or aerosol. Further, such compositions may be administered parenterally, such as by subcutaneous injection, intravenously, intramuscularly, intrasternally, or by various infusion techniques.

Analgesic compositions of the presently disclosed subject matter include, for example and without limitation, analgesically-effective amounts of active agents described herein, and may contain between about 0.01 mg to about 100 g per unit dose of such agents. In alternative embodiments, the amount is between about 0.1 mg to about 10 g per unit dose, or between about 1 mg to about 1 g per unit dose, or between about 1 mg to about 100 mg per unit dose. For the tea tree oil within the analgesic compositions of the presently disclosed subject matter, analgesically-effective amounts may include, without limitation, 0.1 ml to 1 ml of tea tree oil, including 0.15 ml, 0.2 ml, 0.25 ml, 0.3 ml, 0.35 ml, 0.4 ml, 0.45 ml, 0.5 ml, 0.55 ml, 0.6 ml, 0.65 ml, 0.7 ml, 0.75 ml, 0.8 ml, 0.85 ml, 0.9 ml, or 0.95 ml of tea tree oil.

Moreover, the amount of active agent(s) that may be combined with carrier materials to produce a single dosage form will vary depending upon the subject treated and the particular mode of administration. For example, a formulation intended for the oral administration of humans may contain from 0.1 mg to 10 g of active agent compounded with an appropriate and convenient amount of carrier material which may vary from about 5 to about 95 percent of the total composition. Dosage unit forms may generally contain between from about 1 mg to about 1 g of an active agent, typically 25 mg, 50 mg, 100 mg, 200 mg, 300 mg, 400 mg, 500 mg, 600 mg, 800 mg, or 1000 mg.

### II. Methods of Treatment

Once formulated, analgesic compositions of the presently disclosed subject matter may be used without limitation for various analgesic uses. For example, such compositions may be used to treat pain and/or discomfort by administration to an individual experiencing pain or discomfort.

For example and without limitation, analgesic compositions of the presently disclosed subject matter may be used to treat pain due to abrasions, rashes, cuts, burns, cold sores in and out of the mouth, canker sores, mouth sores such as those caused by surgery or cancer treatment with chemotherapy or radiation therapy, surgical wounds, diabetic sores or ulcers, and pressure sores. In one embodiment, compositions of the presently disclosed subject matter are impregnated in adhesive bandages to provide relief from the discomfort of sores or blisters. In another embodiment, compositions of the presently disclosed subject matter are applied topically or intramuscularly to provide relief from muscular or joint pain, or relief from neuropathy. Administration routes also include time-released formulations to provide extended or long-range treatment.

In another embodiment of the presently disclosed subject matter, the anesthetic composition is administered to a subject in need thereof in a therapeutically effective amount to treat pain associated with an oral or dental disorder or condition, particularly alveolar osteitis or dental caries.

Alveolar osteitis (also known as “alveolitis sicca dolorosa” or “dry socket”) is the most common complication following a tooth extraction, particularly from extraction of third molars. Other synonymous terms are “localized osteitis”, “postoperative alveolitis”, “alveolalgia”, “septic socket”, “necrotic socket”, “localized osteomyelitis”, and “fibrinolytic alveolitis”. The incidence of alveolar osteitis has been reported as 3-4% following routine dental extractions and ranges from 1% to 45% after the removal of mandibular third molars. The incidence of dry socket is higher in the mandible, occurring up to 10 times more often for mandibular third molars compared to maxillary molars.

Alveolar osteitis is physically characterized by an empty tooth socket with exposed bone surfaces surrounded by inflamed tissue. The denuded alveolar bone results in extreme pain, irradiating from the empty socket, normally to the ipsilateral ear, temporal region or the eye. The clinical presentation of alveolar osteitis includes post-operative pain in and around the extraction site, which increases in severity at any time between 1 and 3 days after the extraction. Post-operative pain is accompanied by a partially or totally disintegrated blood clot within the alveolar socket, with or without halitosis. Pathogenesis of alveolar osteitis appears to result from the conversion of plasminogen to plasmin resulting in fibrinolysis of the blood clot within the extraction socket.

Factors attributed to the disruption in the healing of the extraction wound include trans-operative complications, presence of local infection, bacterial contamination of the socket, experience of the surgeon, contraceptive use, smoking, alcohol intake, and use of local anesthetics with vasoconstrictors. Individuals with diabetes mellitus, hormonal imbalances, antibiotics-induced immunosuppression, chemotherapy-induced immunosuppression, AIDS-related immunosuppression, and radiation therapy-induced immunosuppression are especially susceptible to problems in the healing of the extraction wounds. Other factors such as smoking, excessive trauma to the tissue site, degree of impaction of the third molar, inadequate irrigation during and after extraction, oral conceptive use, timing in the menstrual cycle, use of an anesthetic with a vasoconstrictor, use of corticosteroids preoperatively, extraction-associated surgical trauma, and experience of the oral surgeon all have been identified as probable causes. Additional risk factors include presence of pericorontitis, high pre- and post-operative bacterial counts.

Dental caries is also a common disease process that afflicts a large proportion of the world population. Extensive research indicates that dental caries is the result of a bacterial infection (Loesche (1986) Microbiol. Rev. 50:353-380), but is also influenced by host and dietary factors (Hicks et al. (2003) J. Clin. Pediatr. Dent. 28:47-52). Current research seeks to identify risk factors for caries and to identify natural oral defenses that may protect against or prevent caries development. Salivary defense systems play a significant role in maintaining the health of the oral cavity and preventing caries. These defenses include factors which inhibit or reverse demineralization of exposed tooth surfaces, such as simple mechanical rinsing, buffering action, and calcium phosphate binding proteins, as well as antimicrobial activities including microorganism aggregation and clearance from the oral cavity, immune surveillance, and the secretion of antimicrobial peptides (Van Nieuw Amerongen et al. (2004) Caries Res. 38:247-253).

Severe dental caries is associated with severe pain until the subject is able to receive treatment. Lidocaine is a local anaesthetic of the amide type that was first introduced in the 1940s and is widely used in injection and for local application to mucous membranes. It has rapid onset of action and anesthesia is obtained within a few minutes depending on the site of administration. It has an intermediate duration of action, with an elimination half-life of approximately 90-120 minutes in most subjects. Benzocaine is an ethyl ester of p-Aminobenzoic acid and is used to relieve pain associated with ulcers, wounds, mucous membranes, and various types of oral and dental pain. Normally benzocaine acts only as long as it is in contact with the skin or mucosal surface, with a peak effect occurring within 1 minute after application and lasting for 30 to 60 minutes.

In the methods and compositions of the presently disclosed subject matter, the therapeutically effective amount, or dosage, is generally dependent on the severity and responsiveness of the state of the disorder or condition to be treated, with the course of treatment lasting from several days to several weeks or months, or until cure or improvement are achieved. Optimal dosing schedules can be calculated from measurements of drug accumulation in the body of the subject and on basis of, e.g., the present examples. Persons of ordinary skill can easily determine optimum dosages, dosing methodologies and repetition rates. In general, dosage is calculated according to body weight, and may be given once or more daily, weekly or monthly. Persons of ordinary skill in the art can easily estimate repetition rates for dosing.

As used herein, the term “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disorder or condition or the prophylactic diagnosis or treatment for preventing the onset of a disorder or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disorder or condition. Thus, the terms “subject” and “patient” are used interchangeably herein. Subjects also include animal disease models (e.g., rats or mice used in experiments, and the like).

As described herein, the presently disclosed compositions can be administered to a subject for therapy by various routes of administration, including orally, transmucosally, topically, or other modes of delivery known in the art.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disorder or condition, or one or more symptoms thereof; prevent the advancement of a disorder or condition, cause the regression of a disorder or condition; prevent the recurrence, development, onset or progression of a symptom associated with a disorder or condition, or enhance or improve the prophylactic or therapeutic effect(s) of another therapy. In the compositions and methods of the presently disclosed subject matter, a therapeutically effective amount of a therapeutic agent is an amount that produces analgesia in a subject for an extended period of time (e.g., days to weeks to months). A physician having ordinary skill in the art can readily determine and prescribe the effective amount of the presently disclosed composition required. Accordingly, the dosage range for administration will be adjusted by the physician as necessary, as described more fully elsewhere herein.

### III. Kits

In some embodiments, the presently disclosed subject matter provides a kit for treating pain in a subject in need thereof, the kit comprising any of the analgesic compositions disclosed herein and instructions for administration of the composition to the subject in an amount effective to treat pain in the subject, wherein the pain is associated with an abrasion, rash, cut, burn, cold sore, canker sore, mouth sore caused by surgery or cancer treatment, surgical wound, diabetic sore or ulcer, or pressure sore.

In another embodiment, the presently disclosed subject matter provides a kit for treating pain in a subject in need thereof, the kit comprising any of the analgesic compositions disclosed herein and instructions for administration of the composition to the subject in an amount effective to treat pain in the subject, wherein the pain is associated with an oral or dental disorder or condition. In particular embodiments, the oral or dental disorder or condition is alveolar osteitis or dental caries.

In its simplest form, a kit according to the presently disclosed subject matter comprises a container containing at least one composition according to the presently disclosed subject matter. In other embodiments, the kit may comprise multiple containers, each of which may contain a portion of a composition according to the presently disclosed subject matter and which may be combined to produce the final composition.

The container can be any material suitable for containing a presently disclosed composition or another substance useful in performing a presently disclosed method. Thus, the container may be a vial or carpule. It can be fabricated from any suitable material, such as glass, plastic, metal, or paper or a paper product. In embodiments, it is a glass or plastic carpule or vial that can be sealed, such as by a stopper, a stopper and crimp seal, or a plastic or metal cap. In embodiments, the container comprises a composition according to the presently disclosed subject matter.

In embodiments, the container is provided as a component of a larger unit that typically comprises packaging materials (referred to herein as a kit for simplicity purposes). The presently disclosed kit can include suitable packaging and instructions and/or other information relating to the use of the compositions. Typically, the kit is fabricated from a sturdy material, such as cardboard and plastic, and can contain the instructions or other information printed directly on it. The kit can comprise multiple containers containing the composition of the invention. In such kits, each container can be the same size, and contain the same amount of composition, as each other container, or different containers may be different sizes and/or contain different amounts of compositions or compositions having different constituents. One of skill in the art will immediately appreciate that numerous different configurations of container sizes and contents are envisioned by this invention, and thus not all permutations need be specifically recited herein.

In general, the kit comprises containers to contain the components of the kit, and is considered a single package comprising a combination of containers. Thus, the components are said to be in packaged combination within the kit. In addition to a container containing the composition of the invention, the kit can comprise additional containers containing additional compositions of the invention. Each container may contain enough of the composition as disclosed herein for a single dose of an embodiment of the method of the invention, or it may contain enough for two or more doses. The various containers may contain differing amounts of the presently disclosed compositions. The kit can further comprise some or all of the supplies and materials needed to prepare for and perform a presently disclosed method, such as, but not limited to, syringes, sterile water or a sterile aqueous solution. In some embodiments, the kits comprise one or more liquids to hydrate the compositions of the kits.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

An analgesic composition for the treatment of alveolar osteitis or dental caries was prepared as follows: 1.2 to 5 grams of zinc oxide and/or 1.2 to 5 grams of Benzocaine 20% gel was combined with 4 to 8 drops of tea tree oil (0.2 ml to 0.4 ml tea tree oil), along with suitable carriers. The composition was applied directly to the affected areas of subjects with alveolar osteitis or dental caries to produce analgesia (see Example 2).

### Example 2

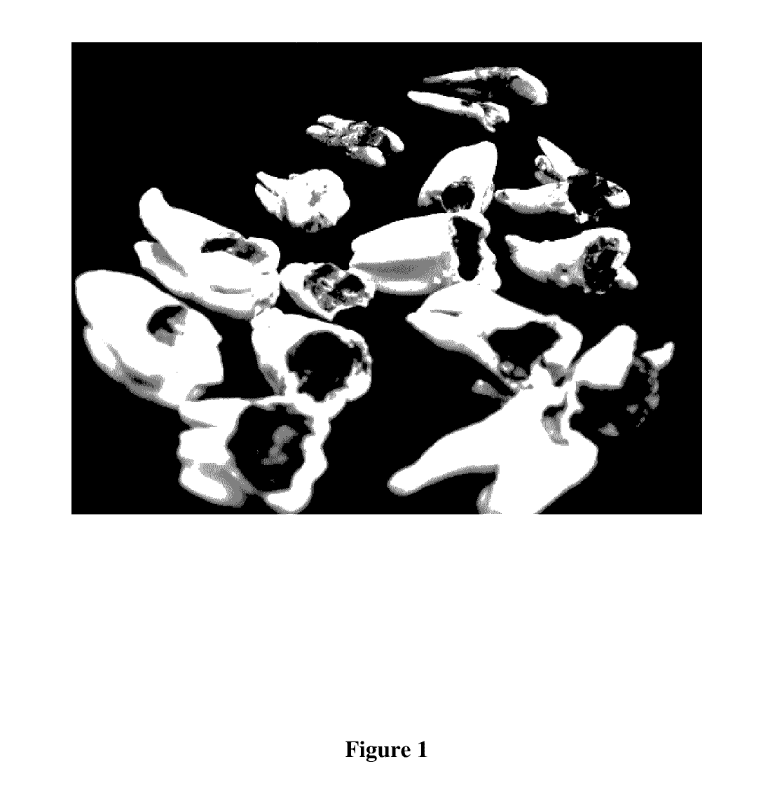

The composition of Example 1 was applied directly to the affected areas of subjects with alveolar osteitis or dental caries. Subjects were approximately 600 adult males, females, and juveniles who had provided informed consent (or whose parents or guardians had provided informed consent). Subjects completed a questionnaire to assess the level of pain they experienced as a result of the alveolar osteitis or dental caries before and after application of the composition of Example 1. A remarkable and surprisingly long-lasting analgesic effect was exhibited by application of the composition of Example 1. The analgesic effect was usually exhibited within less than one minute (approximately about 15 to 45 seconds) following application, and in all cases there was no recurrence for weeks to months. FIG. 1 shows a photograph of teeth with severe dental caries that were removed from patients who received treatment with the composition of Example 1 and reported the rapid onset and long lasting analgesic effect described above.

### Example 3

Tea tree oil was combined with Ginger root oil, Peppermint leaf oil, Chamomile flower oil, Olive leaf extract oil, Grape seed oil, Clove oil, Cherry syrup, and Benzocaine in a ratio of about a 6:1:1:1:1:1:1:1:1 part by weight relationship to form an admixture. All oils were obtained from Esoteric Oils (South Africa). Zinc oxide and water soluble poly(ethyleneglycol) (PEG) such as PEG 3350 and/or PEG 400 was also added to the admixture to form a paste.

### Example 4

An analgesic composition in the form of a gel to be applied to the gums for cleaning and healing gum tissue and numbing for cleaning teeth was prepared as follows: 1 teaspoon or 5 ml PEG 400 was combined with 1.2 to 15 grams of Benzocaine 20% gel and 9 to 15 drops of tree tea oil (0.45 ml to 0.75 ml tea tree oil). The composition is applied directly to the gums of subjects to be treated to produce analgesia.

### Example 5

An analgesic composition for the treatment of canker sores or other mouth sores such as those caused by cancer treatment (e.g., chemotherapy or radiation therapy) was prepared as follows. One teaspoon or 5 ml of ora-sweet was combined with 5 to 10 drops of tree tea oil (0.25 ml to 0.5 ml tea tree oil) and 1.2 to 5 grams of Benzocaine 20% gel. Approximately 1 teaspoon to 1 tablespoon is administered to the mouth of a subject with canker sores or other mouth sores such as those caused by cancer treatment (e.g., chemotherapy or radiation therapy). The subject rinses their mouth with the composition for several minutes (e.g., 3 to 5 minutes) to produce analgesia.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. An anesthetic composition comprising a combination of Tea tree oil and one or more additional agent, comprising 0.1 ml to 1.0 ml of Tea tree oil.

2. The anesthetic composition of claim 1, wherein the ratio of Tea tree oil to the one or more additional agent is a 6:1 part by weight relationship.

3. The composition of claim 1, wherein the additional agent is selected from the group consisting of Ginger root oil, Peppermint leaf oil, Chamomile flower oil, Olive leaf extract oil, Grape seed oil, Clove oil, a flavoring syrup, and an additional anesthetic agent.

4. The composition of claim 3, wherein the flavoring syrup is Cherry syrup.

5. The composition of claim 3, wherein the additional anesthetic agent is a topical anesthetic agent.

6. The composition of claim 5, wherein the topical anesthetic agent is selected from the group consisting of benzocaine, lidocaine, and tetracaine.

7. The composition of claim 5, wherein the topical anesthetic is benzocaine.

8. The composition of claim 2, comprising the Tea tree oil, Ginger root oil, Peppermint leaf oil, Chamomile flower oil, Olive leaf extract oil, Grape seed oil, Clove oil, the flavoring syrup, and an additional anesthetic agent in a ratio of about a 6:1:1:1:1:1:1:1:1 part by weight relationship.

9. The composition of claim 1, wherein the composition further comprises zinc oxide and/or water soluble poly(ethyleneglycol) (PEG).

10. The composition of claim 9, wherein the PEG is PEG 3350 and/or PEG 400.

11. The composition of claim 1, wherein said composition is in a form selected from the group consisting of: an immediate-release composition, a controlled-release composition, sustained-release orally-administrable compositions, topically-administrable compositions, liquid solutions, liquid sprays, lozenges, throat sprays, ointments, solutions, foams, cough drops, dissolvable strips, a jelly, a mouthwash, a gargle, a lollipop, a gum, aqueous or oily suspensions, dispersible powders or granules, a syrup, an elixir, emulsions, a cream, a paste, a gel, a lotion, impregnated dressings, occularly-administrable compositions, inhalable particles, inhalable solutions, droplets, and aerosols.

12. A method for treating pain in a subject in need thereof, the method comprising administering a therapeutically effective amount of the composition of claim 1 to an affected area of the subject, wherein the pain is associated with an abrasion, rash, cut, burn, cold sore, canker sore, mouth sore caused by surgery or cancer treatment, surgical wound, diabetic sore or ulcer, or pressure sore.

13. A method for treating pain in a subject in need thereof, the method comprising administering a therapeutically effective amount of the composition of claim 1 to an affected area of the subject, wherein the pain is associated with an oral or dental disorder or condition.

14. The method of claim 13, wherein the oral or dental disorder or condition is alveolar osteitis.

15. The method of claim 13, wherein the oral or dental disorder or condition is dental caries.

16. A kit for treating pain in a subject in need thereof, the kit comprising the composition of claim 1 and instructions for administration of the composition to the subject in an amount effective to treat pain in the subject, wherein the pain is associated with an abrasion, rash, cut, burn, cold sore, canker sore, mouth sore caused by surgery or cancer treatment, surgical wound, diabetic sore or ulcer, or pressure sore.

17. A kit for treating pain in a subject in need thereof, the kit comprising the composition of claim 1 and instructions for administration of the composition to the subject in an amount effective to treat pain in the subject, wherein the pain is associated with an oral or dental disorder or condition.

18. The kit of claim 17, wherein the oral or dental disorder or condition is alveolar osteitis.

19. The kit of claim 17, wherein the oral or dental disorder or condition is dental caries.
